(19) United States
(12) Patent Application Publication
(10) Pub. No.: US 2021/0100761 A1
(43) Pub. Date: Apr. 8, 2021

(54) MEDICATION TO ELEVATE SOCIAL AND OCCUPATIONAL FUNCTIONING

(71) Applicant: Halo JYSRDB LLC, Dallas, TX (US)

(72) Inventors: Dev Batra, Dallas, TX (US); Shawn Rao, Dallas, TX (US); Joseph Ysbrand, Dallas, TX (US); Jeffrey Hunter, Oklahoma City, OK (US)

(73) Assignee: Halo JYSRDB LLC, Dallas, TX (US)

(21) Appl. No.: 17/038,844

(22) Filed: Sep. 30, 2020

Related U.S. Application Data
(60) Provisional application No. 62/909,504, filed on Oct. 2, 2019.

(51) Int. Cl. A61K 31/192; A61K 31/4178; A61K 31/522; A61K 31/426

(52) U.S. Cl. CPC A61K 31/192; A61K 31/4178; A61K 9/48; A61K 31/426; A61K 31/522

(57) ABSTRACT

The invention is directed to a compositions and methods to improve social and occupational functioning. In particular, the invention is directed to a combination of compounds when taken together improve daily performance levels of social and occupational functioning.

### REFERENCE TO RELATED APPLICATIONS

The present application claims priority to Provisional U.S. Application No. 62/909,504, filed Oct. 2, 2020, entitled “MEDICATION TO ELEVATE SOCIAL AND OCCUPATIONAL FUNCTIONING,” and is incorporated herein in its entirety.

### BACKGROUND

### 1. Field of the Invention

The invention is directed to a compositions and methods to improve social and occupational functioning. In particular, the invention is directed to a combination of compounds when taken together improve daily performance levels of social and occupational functioning.

### 2. Background of the Invention

Medications (also known as medicine, pharmaceuticals, and drugs) are typically used to cure, treat, mitigate, or prevent diseases. Additionally, medications can be used to affect the structure or functioning of the body. Medications may include naturally occurring substances or artificially derived substances.

Common ailments that are treatable by medications include, for example: nausea, vomiting, headache, body aches, stomach pains, fatigue, weakness, thirst, sensitivity to light and sounds, vertigo, decreased attention, decrease concentration, depression, anxiety, irritability, tremors, sweating, increased pulse, and increased blood pressure. Often one medication may be used to treat multiple ailments. However, there is no cure-all and typically multiple medications are necessary to treat simultaneously occurring ailments.

Depending on the severity of the ailments a person is experiencing, the person's social and occupational functioning may decrease. A person experiencing no or mild ailments may have no or little reduction in their ability to perform daily tasks. A person with elevated levels of ailments may have a slight reduction in their ability to perform daily tasks. As the effects of the ailments increases, the person may be less able to complete daily tasks until the ailments get to the point of becoming completely debilitating. Taking multiple medications to alleviate multiple ailments can be expensive, time consuming, and, especially for the elderly, confusing. Therefore, there is a need for a single medication able to simultaneously alleviate the effects of a variety of ailments.

### SUMMARY OF THE INVENTION

The present invention overcomes the problems and disadvantages associated with current treatments and provides new medications.

One embodiment of the invention is directed to a composition containing a combination of medications. The composition comprises a first medication that prevent nausea and/or vomiting, a second medication that decreases the amount of acid the stomach produces, a third medication that ameliorate mild pain, and a fourth medication that provides a central nervous system (CNS) stimulant.

In a preferred embodiment, the first medication comprises ondansetron, meclizine, scopolamine, dimenhydrinate, chlorpromazine, or a combination thereof. Preferably, the second medication comprises a histamine blocker. In a preferred embodiment, the histamine blocker comprises famotidine, nizatidine, cimetidine, ranitidine, calcium, magnesium, or a combination thereof. Preferably, the medication comprises a nonsteroidal anti-inflammatory drug (NSAID). Preferably, the NSAID comprises ibuprofen, aspirin, naproxen, or a combination thereof. In a preferred embodiment, the fourth medication comprises a methylxanthine class of compounds. Preferably, the methylxanthine class of compounds comprises caffeine, amphetamine, ephedrine, or a combination thereof.

Preferably, the composition comprises ondansetron, famotidine, ibuprofen, and caffeine. In a preferred embodiment, the composition comprises at least about 0.7% ondansetron, at least about 3% famotidine, at least about 68% ibuprofen, and at least about 15% caffeine. Preferably the composition further comprises a liquid or powder. In a preferred embodiment the composition further comprises a table or a capsule. The composition preferably further comprises a compounding filler. Preferably, the filler provides for immediate release of the medications. In a preferred embodiment, the filler provides for delayed release of the medications.

Another embodiment of the invention is directed to a method for increasing the Social and Occupational Functioning Assessment Scale (SOFAS) of an individual comprising administering a composition.

Preferably, administration comprises oral administration. In a preferred embodiment, the increase of SOFAS of the individual before and after treatment is at least five points or more. Preferably, the increase of SOFAS of the individual before and after treatment is at least ten points or more. Preferably, the increase of SOFAS of the individual before and after treatment is at least twenty points or more.

Other embodiments and advantages of the invention are set forth in part in the description, which follows, and in part, may be obvious from this description, or may be learned from the practice of the invention.

### DESCRIPTION OF THE FIGURES

FIG. 1 An embodiment of a compounding of a composition of the invention.

FIG. 2 Another embodiment of a compounding of a composition of the invention.

### DESCRIPTION OF THE INVENTION

As embodied and broadly described herein, the disclosures herein provide detailed embodiments of the invention. However, the disclosed embodiments are merely exemplary of the invention that may be embodied in various and alternative forms. Therefore, there is no intent that specific structural and functional details should be limiting, but rather the intention is that they provide a basis for the claims and as a representative basis for teaching one skilled in the art to variously employ the present invention.

A problem in the art capable of being solved by the embodiments of the present invention is multiple ailments may cause a reduction in a person's ability to perform daily tasks, referred to herein as Social and Occupational Functioning. A standard measure of Social and Occupational Functioning is provided by the Social and Occupational Functioning Assessment Scale (SOFAS), which is directed to individual levels of social and occupational functioning. There is no overt correlation to psychological indicators. Thus, a reduction in SOFAS rating is associated with a particular medical condition at a particular time. However, as SOFAS may also be useful to assess additional time frames, such as weeks, months and years prior as the parameters utilized to obtain a SOFAS rating are determinable from prior activities or lack or activities, without including availability and environmental limitations.

The rating of overall psychological functioning on a scale of 0-100 was operationalized in the Health-Sickness Rating Scale. (Luborsky L: “Clinicians' Judgments of Mental Health.” Archives of General Psychiatry 7:407-417 (1962)). (The SOFAS scale was derived from the Global Assessment Scale (GAS) as described in Goldman H H, Skodol A E, Lave T R: “Revising Axis V for DSM-IV: A Review of Measures of Social Functioning.” American Journal of Psychiatry 149:1148-1156 (1992) and Endicott J, Spitzer R L, Fleiss J L, et al.: “The Global Assessment Scale: A Procedure for Measuring Overall Severity of Psychiatric Disturbance.” Archives of General Psychiatry 33:766-771 (1976)). SOFAS values can be characterized as set forth in Table 1

TABLE 1

| | SOFAS |
|---|---|
| Social and Occupational Functioning Assessment | VALUE |
| Superior functioning in a wide range of activities. | 100 |
| Good functioning in all areas, occupationally and socially effective. | 90 |
| No more than a slight impairment in social, occupational, or school functioning | 80 |
| (e.g., infrequent interpersonal conflict, temporarily falling behind in schoolwork). | |
| Some difficulty in social, occupational, or school functioning, but generally | 70 |
| functioning well, has some meaningful interpersonal relationships. | |
| Moderate difficulty in social, occupational, or school functioning | 60 |
| (e.g., few friends, conflicts with peers or co-workers). | |
| Serious impairment in social, occupational, or school functioning | 50 |
| (e.g., no friends, unable to keep a job). | |
| Major impairment in several areas, such as work or school, family relations | 40 |
| (e.g., depressed man avoids friends, neglects family, and is unable to work; | |
| child frequently beats up younger children, is defiant at home, and | |
| is failing at school). | |
| Inability to function in almost all areas | 30 |
| (e.g., stays in bed all day; no job, home, or friends). | |
| Occasionally fails to maintain minimal personal hygiene; | 20 |
| unable to function independently. | |
| Persistent inability to maintain minimal personal hygiene. Unable to function | 10 |
| without harming self or others or without considerable external support | |
| (e.g., nursing care and supervision). | |
| Inadequate information. | 0 |

It has been surprisingly discovered that a composition containing a combination of commercially available medications can measurably increase an individual's SOFAS rating. Increases are at least five points, preferably ten point or more, more preferably twenty points of more, and more preferably thirty points or more. Such improvements are dramatic and represent a substantial increase in overall wellness of the individual as well as health-care professionals and non-professional family members and friends who care for and/or otherwise interact with the individual. The effects of various ailments are greatly reduced, thereby increasing the person's ability to perform daily tasks. In association with improvements of wellness, there are corresponding reductions in expenses, both direct in the way of less medical procedures and medicines, and indirect such as, for example, savings of time.

The composition of the invention preferably contains medications that: (i) prevent nausea and/or vomiting; (ii) decreasing the amount of acid the stomach produces such as a histamine blocker; (iii) ameloriate mild pain such as a nonsteroidal anti-inflammatory drug (NSAID); and (iv) a central nervous system (CNS) stimulant of the methylxanthine class of compounds. For example, the anti-nausea and anti-vomiting drug may be ondansetron, meclizine, scopolamine, dimenhydrinate, chlorpromazine, or another drug. In the preferred embodiment, ondansetron is used in the medication. The histamine blocker medication may be, for example, famotidine, nizatidine, cimetidine, ranitidine, an antacid such as calcium or magnesium, or another drug. In the preferred embodiment, famotidine is included in the medication. The pain reducer and anti-inflammatory drug may be, for example, ibuprofen, aspirin, naproxen, another NSAID, or another drug. In the preferred embodiment, ibuprofen is used in the medication. The stimulant may be, for example, caffeine, amphetamine, ephedrine, or another drug. In the preferred embodiment, caffeine is used in the medication. This composition of the invention is exemplified by the medications ondansetron, famotidine, ibuprofen, and caffeine. It was also surprisingly discovered that compositions of the invention can provide benefits to the individual after only a single treatment, which is unheard of upon administration of any single medication. Multiple treatments also prove beneficial and, accordingly treatments may be daily, every other day, once a week or longer as desired, or twice a day, three times a day, four time a day or more frequently as desired.

Medications are preferable combined in the recommended dose ranges or less, but not in greater amounts. Preferably dose ranges for each medication include 100% or less of the recommended dose, 95% or less or the recommended dose, 90% or less of the recommended dose, 85% or less or the recommended dose, 80% or less of the recommended dose, 75% or less or the recommended dose, 70% or less of the recommended dose, 65% or less or the recommended dose, 60% or less of the recommended dose, 55% or less or the recommended dose, 50% or less of the recommended dose, or various combinations thereof.

Medications may be administered orally, intravenously, subcutaneously, aurally, nasally, or through other methods, and are preferably administered as a combination. Oral methods of administering medication include, but are not limited to, capsules, tablets, liquids, and aerosols. In the preferred embodiment, the medication is administered by a capsule or tablet. Preferably, the drugs are in a powdered form (e.g., lyophilized) and are mixed together as a composition prior to being formed into a tablet or added to a capsule. In some embodiments, a compounding filler agent (such as LOXORAL, lactose, or starch) may be used to increase the volume of the medication, add color, flavor, or texture, or provide other physical properties or attributes to the medication that are not present in the drugs alone. Preferably, the filler has no therapeutic effect. Depending on the size, shape, and makeup of the delivery device, different amounts of filler may be used. The filler allows for immediate release of the medication or for controlled release of the medication, or delayed release over a period of time.

A preferred composition of the invention contains at least about 0.5% ondansetron, at least about 3% famotidine, at least about 68% ibuprofen, and at least about 15% caffeine. More preferably, the medication has between 0.25-2% ondansetron, between 2-5% famotidine, between 60-75% ibuprofen, and between 10-20% caffeine. The remaining amount of the medication may be another drug or filler.

Preferably, a single dose of the medication is administered to the patient. However, depending on the severity of the patient's ailments, the patient's weight, height, gender, age, and/or other demographics, multiple doses of medication may be administered. The doses may be administered all at once or over time. Preferably, upon alleviation of the ailments, no further doses are administered.

The following examples illustrate embodiments of the invention, but should not be viewed as limiting the scope of the invention.

### EXAMPLES

### Example 1

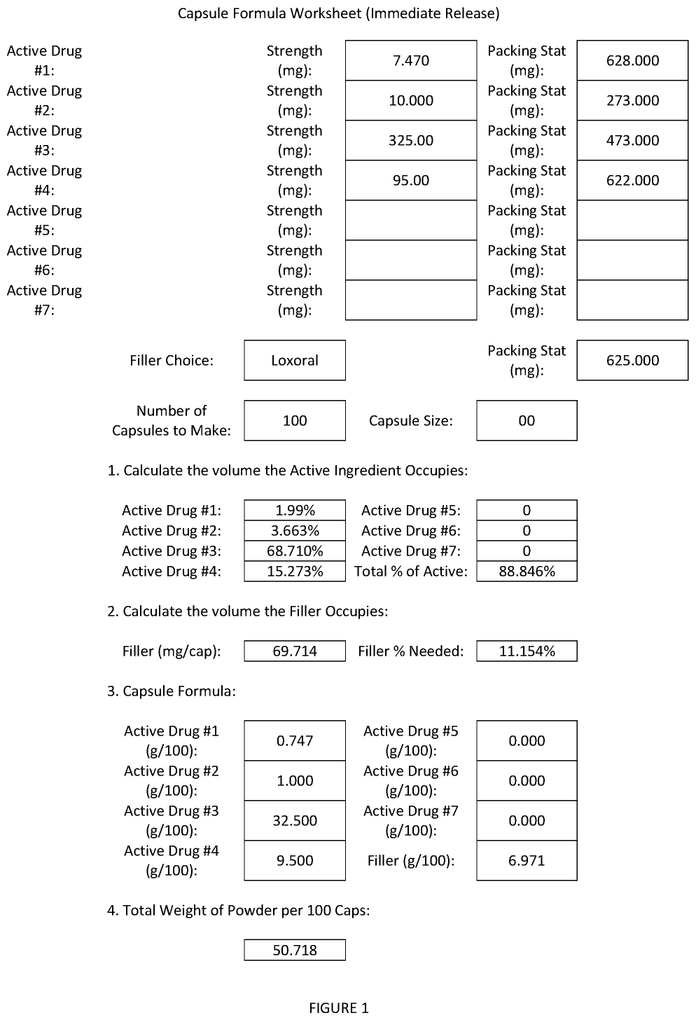

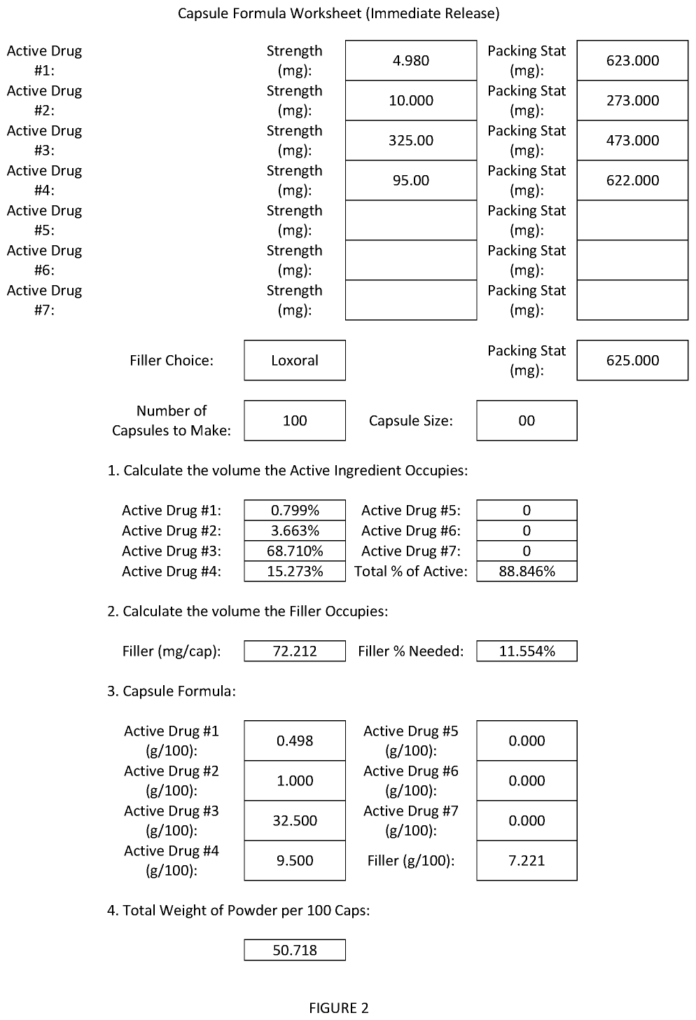

FIG. 1 provides a summary of the compounding of a composition of one embodiment of the invention. FIG. 2 provides a summary of the compounding of a composition of another embodiment of the invention.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only with the true scope and spirit of the invention indicated by the following claims. Furthermore, the term “comprising of” includes the terms “consisting of” and “consisting essentially of.”

1. A composition containing a combination of medications comprises of:
a first medication that prevent nausea and/or vomiting;
a second medication that decreases the amount of acid the stomach produces;
a third medication that ameliorate mild pain; and
a fourth medication that provides a central nervous system (CNS) stimulant.

2. The composition of claim 1, wherein the first medication comprises ondansetron, meclizine, scopolamine, dimenhydrinate, chlorpromazine, or a combination thereof.

3. The composition of claim 1, wherein the second medication comprises a histamine blocker.

4. The composition of claim 3, wherein the histamine blocker comprises famotidine, nizatidine, cimetidine, ranitidine, calcium, magnesium, or a combination thereof.

5. The composition of claim 1, wherein the third medication comprises a nonsteroidal anti-inflammatory drug (NSAID).

6. The composition of claim 5, wherein the NSAID comprises ibuprofen, aspirin, naproxen, or a combination thereof.

7. The composition of claim 1, wherein the fourth medication comprises a methylxanthine class of compounds.

8. The composition of claim 7, wherein the methylxanthine class of compounds comprises caffeine, amphetamine, ephedrine, or a combination thereof.

9. The composition of claim 1, which comprises ondansetron, famotidine, ibuprofen, and caffeine.

10. The composition of claim 9, which comprises at least about 0.7% ondansetron, at least about 3% famotidine, at least about 68% ibuprofen, and at least about 15% caffeine.

11. The composition of claim 1, which comprises a liquid or powder.

12. The composition of claim 1, which comprises a table or a capsule.

13. The composition of claim 1, further comprising a compounding filler.

14. The composition of claim 13, wherein the filler provides for immediate release of the medications.

15. The composition of claim 13, wherein the filler provides for delayed release of the medications.

16. A method for increasing the Social and Occupational Functioning Assessment Scale (SOFAS) of an individual comprising administering the composition of claim 1.

17. The method of claim 16, wherein administration comprises oral administration.

18. The method of claim 16, wherein the increase of SOFAS of the individual before and after treatment is at least five points or more.

19. The method of claim 16, wherein the increase of SOFAS of the individual before and after treatment is at least ten points or more.

20. The method of claim 16, wherein the increase of SOFAS of the individual before and after treatment is at least twenty points or more.
